(19) United States
(12) Patent Application Publication
(10) Pub. No.: US 2021/0187314 A1
(43) Pub. Date: Jun. 24, 2021

(54) METHOD AND SYSTEM OF DNA RESONANCE AND ITS APPLICATIONS

(71) Applicants: Max Myakishev-Rempel, San Diego, CA (US); Alexandre Perry Kamel, Denver, CO (US)

(72) Inventors: Max Myakishev-Rempel, San Diego, CA (US); Alexandre Perry Kamel, Denver, CO (US)

(21) Appl. No.: 16/721,097

(22) Filed: Dec. 19, 2019

(51) Int. Cl. A61N 2/00; A61N 2/02

(52) U.S. Cl. CPC A61N 2/004; A61N 2/02

(57) ABSTRACT

Method and system of DNA resonance and its applications which is done non-chemically using impedance spectroscopy and analyzing it to produce the diagnostic profile of the health and using initial treatments to see how the profile change and then adjusting the treatment to improve the profile in a specific direction, adjusting the parameters of the electric, electromagnetic and sound treatment for better outcomes. The initial treatment is at 42.2 Gigahertz, which leads to Alu sequence decompaction and activation of the DNA leading to improved biological outcomes, improved coherence, and orderliness of the DNA and of the DNA fractal patterns. Further, the present invention is intended to various applications such as in therapy, communication to and from the body, communication to and from the brain, biotechnology and biological research using electromagnetic waves, patterned electric voltage, magnetic pulses, sound or other such means of stimulation applied individually and/or in combinations.

### FIELD OF INVENTION

This invention relates generally to the field of biomedical engineering and is particularly directed to a method and system of DNA resonance and its applications. More specifically, the present invention comprises of a method and system of DNA resonance measurement which is done non-chemically.

### BACKGROUND OF THE INVENTION

DNA resonance treatments can be applied to therapy, performance improvement, performance improvement in fitness, research instrumentation, biotechnological production, food industry, agriculture, for brain-computer interface (bidirectional exchange of information), use for synthetic telepathy, use for education, for the manipulation of the memory, for therapy of psychiatric disorders, for manipulation of the mind, for medical and non-medical adjustment of mood, emotions, sleep, brain activity, meditation, mental performance, studying performance, work performance, performance in unfavorable conditions, sports, fight, for performance under stress and for stress reduction.

### DISCLOSURE OF PRIOR ART

A patent search conducted showed several patented inventions that are related to the art.

The concept of a DNA resonance and the application of this technology is already known. There are available patents which described the same. For instance, a patented invention described a method for readily and efficiently determining resonant frequencies that can be used therapeutically or beneficially, for debilitation of specific types of genomic materials, including DNA and/or RNA, genes, and gene sections. The methods can be used in a variety of circumstances related to various human and animal diseases and conditions. Methods allow the determination of therapeutic resonant frequencies for use in media having various refraction properties (U.S. Pat. No. 7,280,874).

Meanwhile, another patent disclosed according to some embodiments, an electromagnetic resonance-based disease treatment system that comprises a processing unit configured to generate a resonant frequency signal and a radiating antenna configured to radiate an electromagnetic field based on the resonant frequency signal. The resonant frequency signal may carry at least one frequency at which reference materials related to a disease condition resonate (U.S. Pat. No. 9,610,458).

Another disclosure also relates, according to some embodiments, to an electromagnetic resonance-based disease treatment system that comprises a processing unit configured to generate a resonant frequency signal and a radiating antenna configured to radiate an electromagnetic field based on the resonant frequency signal. The resonant frequency signal may carry at least one frequency at which reference materials related to a disease condition resonate. An antenna configuration may be used to determine one or more resonant frequencies of the reference materials. A subject having or at risk of having the disease condition may be exposed to the electromagnetic field in order to treat the disease condition (WO2018005031A1).

In another disclosure, tumors can be treated with an alternating electric field. The size of cells in the tumor is determined prior to the start of treatment by, for example, biopsy or by inverse electric impedance tomography. A treatment frequency is chosen based on the determined cell size. The cell size can be determined during the course of treatment and the treatment frequency is adjusted to reflect changes in the cell size. A suitable apparatus for this purpose includes a device for measuring the tumor impedance, an AC signal generator with a controllable output frequency, a processor for estimating the size of tumor cells and setting the frequency of the AC signal generator based thereon, and at least one pair of electrodes operatively connected to the AC signal generator such that an alternating electric field is applied to the tumor (US20140330268A1).

Further, another present relates to a pharmaceutical composition, kit or combined preparation comprising benzo[e]pyrido indoles and DNA-damaging agents, said composition, kit or combined preparation being useful for treating cancer. It also relates to the combined treatment of cancer with benzo[e]pyrido indoles and radiotherapy (WO2011131636A1).

Also, the treatment of cancer through electronically chemical infusion was presented in another patent, using a newly adaptive language of bio/signaling cellular communications. This study presents an effective cancer treatment via solar sonic electromagnetic chemical which is infusionably accessible and comprehensively coded for a variety of cancer conditions. All cancerous toxic components will undergo a form of physiological/genetic/reverse engineering. All potential pharmacological toxicity will be chemically reformulated and recondensed to specifically penetrate carcinogenic cells and lead to gradual eradication without affecting healthy cells, blood components or other internal organs. This invention relates to a medical device specially designed to treat cancer through a mechanism of promoting cytostasis, impairing mitosis, infusing cytochrome C-Protein and programming T-Lymphocyte/T-Cells, as well as B-lymphocyte/B-cells, thymus gland and inducing apoptosis; all done simultaneously with a medicament selected from highly proprietary pharmaceutically infused therapeutic composition and immunotherapeutic agents, immunomodulators, and—antigenic agents, angiogenesis inhibitors, vascular sealing agents, gene-therapy agents, antibiotic-resistant modification agents and a photodynamic therapy agents (WO2016097831A1).

In another invention, cells that are in the process division are vulnerable to damage by AC electric fields that have specific frequency and field strength characteristics. The selective destruction of rapidly dividing cells can, therefore, be accomplished by imposing an AC electric field in a target region for extended periods of time at particular frequencies with particular filed strengths. Some of the cells that divide while the field is applied will be damaged, but the cells that do not divide will not be harmed. This selectively damages rapidly dividing cells like bacteria but does not harm normal cells that are not dividing. Since the vulnerability of the dividing cells is strongly related to the alignment between the long axis of the dividing cells and the lines of force of the electric field, improved results can be obtained when the field is sequentially imposed in different directions (US20110137229A1).

Moreover, another patent presented a binodal lemniscate crown that alters abnormal brain synchrony patterns observed in autistic individuals establishing a harmonized flow of electromagnetic energy in the neural pathways that provides relief for headaches, reduces or eliminates various symptoms of autism, enhances memory and ability to focus; the said crown consisting of three conjoined circles that are formed from a piece of copper wire by bending it (CA Patent No. 2682098A1).

The WO Patent No. 1999002217A1 also described an invention that relates to the medical field, and in particular to impulse magneto therapy of biological objects. The therapeutic effect is achieved by concurrently subjecting the organism or/and the treated organ of the patient to the acoustic effect. As the acoustic effect there is applied, harmonious sounding producing a stimulating effect. Furthermore, another patent also presented an apparatus and a method for therapeutically treating human body tissue with electromagnetic radiation (U.S. Pat. No. 5,723,001).

Moreover, the U.S. Pat. No. 6,770,042 B2 also disclosed an invention for treating diseases with resonance generating electromagnetic fields. Multiple frequency bands with nonlinear frequency variation are combined to enhance the effectiveness of the therapy delivered by an infrasonic transducer or another delivery mechanism.

Another patent also relates, according to some embodiments, to an electromagnetic resonance-based disease treatment system that comprises a processing unit configured to generate a resonant frequency signal and a radiating antenna configured to radiate an electromagnetic field based on the resonant frequency signal. The resonant frequency signal may carry at least one frequency at which reference materials related to a disease condition resonate. An antenna configuration may be used to determine one or more resonant frequencies of the reference materials. A subject having or at risk of having the disease condition may be exposed to the electromagnetic field in order to treat the disease condition (WO2018005031A1).

In the field of disease detection, another invention discloses a microdevice for detecting or treating a disease, each device comprising a first sorting unit capable of detecting the properties of a biological sample at a microscopic level and sorting the biological sample according to the detected property; the first detecting unit. It is capable of detecting an identical or different property of the sorted biological sample at the microscopic level; and the first layer of material comprising an outer surface and an inner surface, the inner surface defining a first channel in which the biological sample is from A sorting unit is transported to the first detecting unit (TWI618932B).

Magnetic field generating device and method of generating and applying a magnetic field for treatment of specified conditions was also disclosed in another patent. A magnetic field generator device includes a signal generator capable of operating within predetermined parameters. An attenuator is connected to the signal generator and to Helmholtz coils to transmit, and attenuate the signal from the generator to the coils to generate a predetermined desired magnetic field. A method of treating patients having numerous conditions provides for subjecting the patients, patient portions and/or targets therein, to the magnetic fields which are set in accordance with the characteristics of the patient, patient portions, and/or target to be treated (WO2000013749A1).

Another patent also described a system for the electromagnetic treatment of mammals has a signal generator for generating an electromagnetic signal, an organic filter for supplying the desired spectrum of electromagnetic signals, and a radiating antenna for radiating the desired spectrum. In a method of treating a mammal, an electromagnetic signal is generated, the generated signal is supplied to an organic filter to provide signals with the desired spectrum and the desired spectrum of signals is radiated into a space occupied by the mammal to be treated. A feedback antenna receives the radiated signals and provides a feedback signal which is amplified and supplied to the filter. In particular, the filter may have a driven antenna, a filter element containing a biological sample and a receiving antenna in a shielded housing (US20080021526A1).

Meanwhile, the treatment or amelioration of arthritic joint pain using the electromagnetic field was also presented in another patent. An apparatus for treatment of pain includes a coil formed by a generally flat elliptical base member wrapped by wire in two separated grooves spaced axially along the outer surface and a portable battery-powered generator unit for generating a current such that the electromagnetic field has a required magnitude of magnetic flux density in the range 1×104-6 gauss to 1×10-12 gauss at a frequency of between 0.01 and 100 Hz. The base member of the coil is covered by a flexible fabric covering material with a padding layer on each side. The coil is elliptical in order to generate a field of the required magnitude emitted outwardly to one side in an area of the order of 10 inches by 5 inches at a depth of 2 inches. The generator is controlled wholly by simple switches which only select from a plurality of treatment options (US20100113862A1).

Furthermore, another patent disclosed a therapeutic method and apparatus to induce the healing of tissue cells by an electrical stimulus to a tissue area with the problematic condition by the application of low-density charges of alternate polarities. The apparatus performs the therapeutic function by alternately charging and discharging the cells in 15-minute intervals over periods of time from 1 to 12 hours (U.S. Pat. No. 6,016,450).

Another patent presented an apparatus which comprises an electromagnetic radiation source with periodic emission, associated with a filter which, under conditions of use, is located between the source and the epidermis of the patient. This has an application to improving the receptivity of a patient to therapeutic treatments (FR2607016A1).

A method and apparatus for ameliorating the aging process and the effects of aging and maintaining the integrity of health were also disclosed. The method includes subjecting biological systems to alternating and steady magnetic fields having flux densities ranging from 10-6 gauss to 10-20 gauss and frequencies from 0 Hertz to 1014 Hertz. The calculation is made with reference to the equation mc2=Bvlq, where m=mass; c=speed of light; B=magnetic flux density; v=inertial velocity of the mass contained in I; l=length of the conductive body; q=unity. The process begins by targeting the larger targets first and then diminishing the field magnitude slowly and incrementally according to the targets. The frequency when AC is indicated is calculated with the cyclotron resonance formula, fc=qB/(2 πm). The apparatus includes a specially constructed pool or tub for generating the specific magnetic flux necessary for treatment. The orientation of the patient with reference to North, South, East, and West is varied. The earth's position in relation to the sun is taken into account. The patient may be in an upright, prone or swimming position depending on the specific treatment scheme (U.S. Pat. No. 6,004,257A).

A method for repair and regeneration of bone marrow using DNA resonance was also presented. The invention is directed to a method for treating tissue or organ in a subject by directly administering an effective amount of an exogenous, decellularized extracellular matrix or a mixture of extracellular matrix and mesenchymal stem cells into the intended site of activity, such as bone marrow cavity. In one embodiment, the invention provides methods of treating bone marrow to increase the number of circulating progenitor and stem cells. In some other embodiments of the invention, the decellularized extracellular matrix to be directly administered is configured to be a time-released therapeutic (US20090180965A1).

In another invention, systems and methods for providing a magnetic resonance treatment to a subject were presented. A system for providing a magnetic resonance treatment may include components that provide the system with an ability to treat pain and relieve symptoms of the subject. In another embodiment, a method of providing a magnetic resonance treatment includes components that provide the system with an ability to treat pain, relieve symptoms, provide relaxation, and improve the overall comfort and well-being of the subject (U.S. Pat. No. 9,724,534B2).

Another patent also presented a method and apparatus for non-invasive therapy of cardiovascular ailments using weak pulsed electromagnetic radiation. A method and an apparatus for the treatment of cardiac hypertrophic heart failure, hypertrophic cardiomyopathy, atrial or ventricular bradyarrhythmia (slow heart rate), atrial flutter-fibrillation and similar cardiac ailments, as well as peripheral vascular disease and hypertension, using a weak pulsed magnetic field or a very weak magnetic field. A transducer that emits weak electromagnetic radiation is placed on the patient's chest or legs and, as a result, the very weak electromagnetic field can cause activation, reactivation, inhibition or remodeling of electrophysiological change in cardiac tissue in an irradiated heart or vessels. This treatment method has a wide application for use in patients with various heart and vascular ailments (US20050222625A1).

Meanwhile, another patent described a method for using resonant acoustic and/or resonant acousto-EM energy to detect and/or effect structures. The invention utilizes a biological or inorganic form detection and/or identification and Resonance sound and/or acousto-EM energy applied to the structure in order to increase and/or destruction of the functions in the biological structure. More specifically, the present invention provides a virus, bacteria, fungi, insects, and to a method for the detection and destruction of biological structures, such as oncogenic resonant acoustic and or acoustic energy −EM in biological structures. Moreover, the present invention generates a resonant sound and/or acoustic energy −EM in biological structures, such as a bone by providing a method of increasing the function. 0 people acoustic and/or system for the generation and detection of resonance sound −EM energy is also provided (KR20010090725A).

Another patent also described a DNA Telomere rejuvenation. A copper screen placed between two one inch foam pads and covered with crushed sapphire crystal is connected to an activated Tesla coil. This leads to increased telomere length in normal white blood cells and can be used to increase the reproductive capacity of cells and to delay the onset of cellular senescence (US Patent No. 2011/0077727).

Another patent also described a device, method and system for the microwave resonance therapy of chromosomes, telomeres and DNA and extending the length of telomeres, by generating a wide and uniformly distributed spectrum of electromagnetic frequencies and ultrasound vibrations that induce electromagnetic and ultrasonic resonance in chromosomes and telomeres and electromagnetic resonance in DNA. Moreover, another patent presented an invention which is a method and apparatus for applying low energy, non-ionizing, non-thermal electromagnetic radiation or electric current to the body of the subject for therapeutic and health-promoting purposes (WO Patent No. 00/15295).

Another patent also described a resonance system for use on a subject having a biological pathology. The system includes a wave generator operably disposed adjacent a subject for generating a wave (which can be an electric wave) at a predetermined frequency, a resonant frequency sensor and generator operably disposed adjacent the subject for sensing a resonant frequency of a predetermined area of the subject in response to the predetermined frequency and generating a resonant frequency signal in response to said sensed resonant frequency, and a device (which is preferably computer-based) operably associated with the sensor for receiving the resonant frequency signal and manipulating the resonant frequency signal in a manner to be displayed (US Patent No. 20040068168A1).

Another method was also disclosed intended for measuring in real-time the frequency and the power of an electromagnetic (EM) field emitted by a human body for different body conditions, the resonance phenomenon between the EM fields emitted by various products or electronic devices and the EM field emitted by a human body, and the signals emitted by active materials, waters, topical products, etc. A first real-time spectrum analyzer is connected to a broad frequency range antenna placed in contact with the skin, electromagnetic fields emitted by a particular condition of the subject human body are measured to reveal peaks of power, and a second narrow frequency antenna is used to measure the peak more accurately (WO2010039465A2).

Furthermore, provided in another invention is a remote sensing apparatus comprising: an electromagnetic field detector and an acoustic resonator comprising an electromagnetic field generator and a sensing material in wireless communication with the generator; wherein the sensing material is in wireless communication with the detector, and an acoustic property of the sensing material is responsive to a change in state of an environment to which the sensing material is exposed, and wherein the sensing material is in the form of one or more particles and/or fragments (EP1842051A1).

Another patent also described an application of surface plasmon resonance technology for detecting and genotyping HPV. The present invention discloses using SPR technology to simultaneously and qualitatively detect different HPV genotypes. It also discloses an efficient formula to make a mixed SAM that can greatly enhance the immobilization ability of the metal surface in SPR based techniques, which is good for the immobilization of HPV specific DNA probes used for the detection of different HPV genotypes (WO2009045216A1).

Magnetic resonance system and method to detect and confirm analytes were also disclosed. A system and method are provided to detect target analytes based on magnetic resonance measurements. Magnetic structures produce distinct magnetic field regions having a size comparable to the analyte. When the analyte is bound in those regions, magnetic resonance signals from the sample are changed, leading to the detection of the analyte (US20070166730A1).

Another patent also present an apparatus which can enhance sensitivity, specificity, efficiency, convenience, and rate at low cost in detection of early circulating tumor cells The present invention provides a device comprising a micro-filter, a shutter, a cell counter, a selector, a microsurgical kit, a timer, and a data processing circuitry, the micro-filter being capable of filtering circulating tumor cells by an electrical, magnetic, electromagnetic, thermal, optical, acoustical, biological, chemical, electro-mechanical, electro-chemical, electro-optical, electro-thermal, electro-chemical-mechanical, bio-chemical, bio-mechanical, bio-optical, bio-thermal, bio-physical, bio-electromechanical, bio-electro-chemical, bio-electro-optical, bio-electro-thermal, bio-mechanical-optical, bio-mechanical thermal, bio-thermal-optical, bio-electro-chemical-optical, bio-electro-mechanical-optical, bio-electro-thermal-optical, bio-electro-chemical-mechanical, physical, or mechanical property, or a combination thereof (JP2018148925A).

Described in another patent are a system and method that “reads” cancer tumors real-time and custom delivers individualized bioelectric therapy to the patient. For example, the system reads a cancer tumor, and based upon this reading, delivers to the subject “a confounding signal” to jam communication within that tumor. A cancer tumor may change its communication patterns and the therapy is designed to change with these patterns, attempting to always jam the relevant communication signaling pathway. The described system includes parameters not tied to communication jamming, which should also be customized to induce apoptosis to the cancer tumor. Such parameters include signals for starving a cancer tumor of blood supply and signals for changing the cancer tumor's surface proteins and/or charge so that the immune system attacks the cancer tumor (US20190030330A1).

Another disclosure provide a system and method are provided to detect target analytes based on magnetic resonance measurements. Magnetic structures produce distinct magnetic field regions having a size comparable to the analyte. When the analyte is bound in those regions, magnetic resonance signals from the sample are changed, leading to the detection of the analyte (US20070166730A1).

Another invention described a method and apparatus for the detection of biological molecules. A detection system and a method for the detection of a plurality of substances are disclosed. The detection system has a plurality of detection probes, each of the detection probes having an up-conversion fluorescing core of dimensions less than 200 nm and is linked to an affinity moiety. The affinity moiety bonds to one of the plurality of substances (US20060223197A1).

Further, a resonant magnetic disk for bioanalyte detection was also disclosed in another invention. Embodiments of the invention relate generally to ferromagnetic microdisks, methods of detecting target bioanalyte using ferromagnetic microdisks, and kits (such as for use in the laboratory setting) containing the reagents necessary to make, and/or use ferromagnetic microdisks for bioanalyte detection, depending on the user's planned application. The methods and products allow the fabrication of ferromagnetic microdisks, and their use in the detection of biological molecules with high sensitivity, little or no signal decay, improved safety, convenience, and lowered cost for use and disposal (U.S. Pat. No. 8,445,294B2).

While there are available patents that described DNA resonance applications, most of these inventions are focused on treating diseases with resonance generating electrical or electromagnetic fields. There has been no method and system that provides a device which is non-destructively affecting DNA and/or receiving information from DNA in live cells via non-chemical means. Moreover, an algorithm for the conversion of DNA sequence into the wave patterns has not also been described in available patents. Apparently, this system and method applied for a patent are new to the art and are found innovative which proves useful in the field of DNA resonance and applications. The present invention is directed towards this new and innovative idea.

Referring to “non-chemical cell-cell communication” effects, for instance, most basic experiments on biological fields involved two samples such as cell culture aliquots in sealed quartz cuvettes separated by optical filters. When one of the aliquotes is perturbed, the second one may catch the signal that is transferred non-chemically and is blocked by light impermeable filters. This refers to “non-chemical cell-cell communication” effects.

Meanwhile, although the idea that the morphogenic field is holographic and is produced by the genomic sequence has been around since 1973 (Miller et al., 2012), there have been no attempts to decipher the algorithm of the conversion of DNA sequence into the wave patterns. Here, we name this algorithm as “DNA resonance code” and define it as an algorithm that describes the conversion of genomic DNA sequence into the structure of the morphogenic field and ultimately to the shape of the body. We believe that the DNA resonance code is not only the algorithm that reads the DNA structure and converts it into the wave structure, but that the process is two-directional—the genome receives the information via wave resonance and converts it into the structural information of the DNA by epigenetically condensing and de-condensing chromatin, modifying chromatin's chemistry and thus recording the received wave signals into the chemical structure.

It is therefore an objective of the present invention to provide a unique method and system of DNA resonance and its applications. In particular, a device that is non-destructively affecting DNA and/or receiving information from DNA in live cells via non-chemical means. Applications of the device to therapy, communication to and from the body, communication to and from the brain, and biotechnology and biological research using electromagnetic waves, electric voltage varied in time, magnetic pulses, sound or other such means of stimulation applied individually and/or in combinations. Moreover, by providing an algorithm of the conversion of DNA sequence into the wave patterns.

In this invention, the initial treatment is at 42.2 Gigahertz, which leads to Alu sequence decompaction and activation of the DNA leading to improved biological outcomes, improved coherence, and orderliness of the DNA and of the DNA fractal patterns.

### OBJECTIVES OF THE INVENTION

The general purpose of the present invention is to provide a unique method and system of DNA resonance and its applications.

One significant objective of the present invention is to provide a method and system of DNA resonance using an impedance spectroscopy and analyzing it to produce the diagnostic profile of an individual's health and using initial treatments to see how the profile change and then adjusting the treatment to improve the profile in a specific direction, adjusting the parameters of the electric, electromagnetic and sound treatment for better outcomes

Another objective of the present invention is to provide a method and system of DNA resonance intended to various applications such as in therapy, communication to and from the body, communication to and from the brain, biotechnology and biological research using electromagnetic waves, patterned electric voltage, magnetic pulses, sound or other such means of stimulation applied individually and/or in combinations.

Yet another significant objective of the present invention is to provide an algorithm for the conversion of DNA sequence into and from the wave patterns.

These and other objects and features of the present invention will be apparent from the following detailed description, taken with reference to the figures of the accompanying drawing.

### SUMMARY OF THE INVENTION

The present invention provides a method and system of DNA resonance and its applications. In particular, a device that is non-destructively affecting DNA and/or receiving information from DNA in live cells via non-chemical means.

Applications of the device presented in this invention to therapy, communication to and from the body, communication to and from the brain, and biotechnology and biological research using electromagnetic waves, electric voltage varied in time, magnetic pulses, sound or other such means of stimulation applied individually and/or in combinations.

In DNA resonance measurement, the information is obtained electromagnetically, electrically, acoustically, opto-acoustically or electro-acoustically. DNA resonance measurements can be utilized for diagnostics, non-diagnostics information gathering, identification of humans and livestock, or pets identifying those of the status and identity to an individual readout of internal processes and by the technological process.

This invention can be applied in the treatment of various infectious diseases, infectious and non-infectious disorders, cancers, brain-related and psychological conditions, including dementia, Alzheimer's. Autism, Schizophrenia, depression, and various forms of addiction as well as mechanical injury and physical trauma, and regenerative therapies including organ regeneration or repair, and/or brain-computer interface (bidirectional exchange of information), and for synthetic telepathy. These are indicative, and not intended to be exhaustive with regard to fields of beneficial application.

### BRIEF DESCRIPTION OF THE DRAWINGS

A clear understanding of the key features of the invention summarized above may be had by reference to the appended drawings, which illustrate the method and system of the invention, although it will be understood that such figures depict preferred embodiments of the invention and, therefore, are not to be considered as limiting its scope with regard to other embodiments which the invention is capable of contemplating. Accordingly:

FIG. 1 shows that the microtubules and DNA communicate resonantly through the nuclear membrane.

FIG. 2 illustrates that in neurons, the propagation of action potential across the axons is complemented and possibly is coordinated with the signaling via microtubules.

FIG. 3 is a cross-sectional view of the device of the present invention.

FIG. 4 is a perspective view of the oscillation model for the present invention Cdc25a promoter.
(A) A map of the Cdc25a gene promoter (to scale). Only the beginning of the gene is shown.
(B) A flattened view of the cylindrical surface of (G). The straight horizontal thick black line shows that DNA is in its natural B-form, without additional twisting. The hatched boxes correspond to the repeats and the gene in (A).
(C-F and H) Relative twisting oscillation of the double helix of the Cdc25a promoter.
(C-F) A flattened view of phases of the twisting oscillation
(C) The initial phase of the oscillation, (GGGA)8 is untwisted by a pulse of 850 nm irradiation.
(D) The opposite, second phase of the oscillation: after the restoration of the original state of DNA, (GA)28 is untwisted by a pulse of 660 nm irradiation.
(E) A combination of Images B and C.
(F) Repeated alternation of 660 nm and 850 nm pulses leads to oscillations that propagate outwards reaching Cdc25a transcription start (to activate transcription).
(G) 3D projection of the double helix wrapped in a cylinder.
(H) 3D projection of twisting oscillation.

FIG. 5 is a schematic illustration of peak phase coordination of alternated (ALT) and syncrhonous (SYN) modes.

### DETAILED DESCRIPTION OF THE INVENTION

The present disclosure is to be considered as an exemplification of the invention and is not intended to limit the invention.

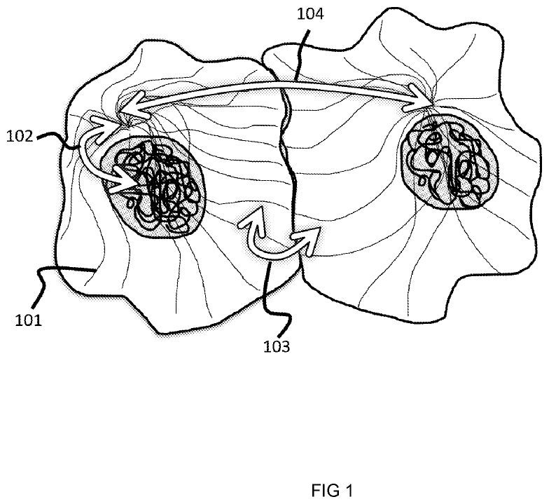

In that form of the present invention chosen for purposes of illustration, FIG. 1 shows the main body of the invention.

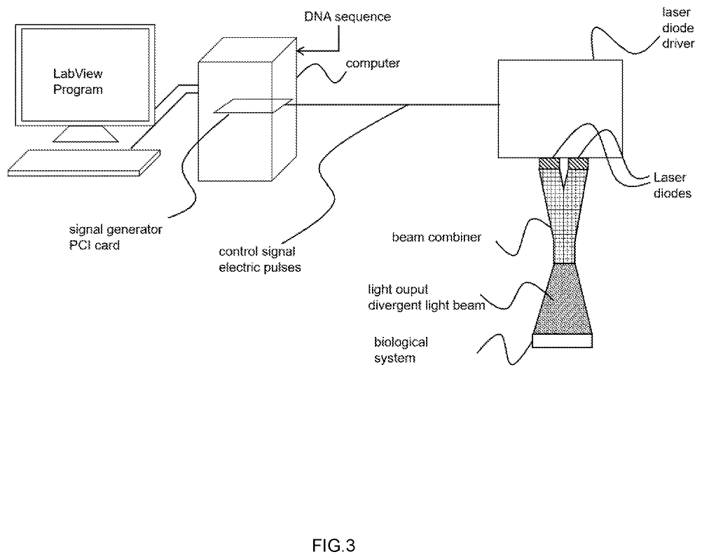

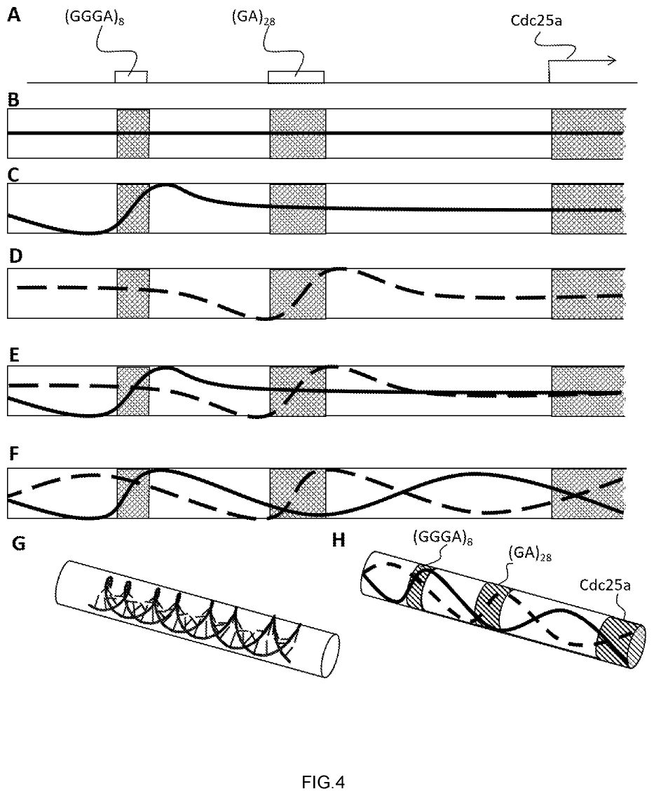

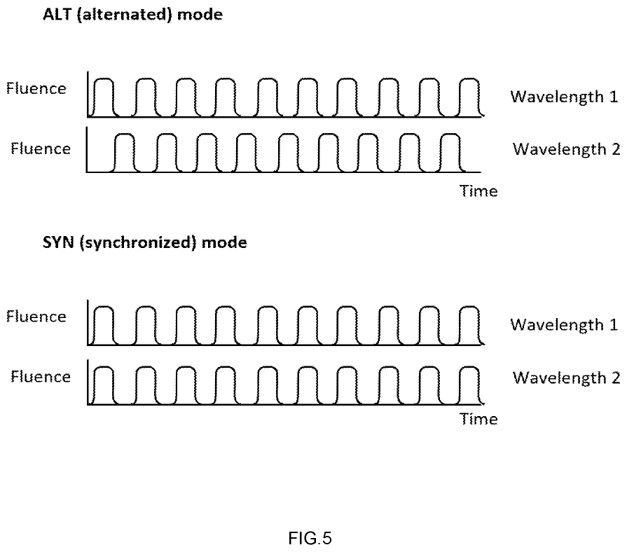

The invention is a method and system of DNA resonance and its applications. In particular, this invention is a device which is non-destructively affecting DNA and/or receiving information from DNA in live cells via non-chemical means. It is also a device where one or more of the following is used: electromagnetic waves, electric voltage varied in time, magnetic pulses, sound or other such means of stimulation applied individually and/or in combinations. As shown in FIG. 3 the present system comprises of
a PC computer,
a Signal generator PCI card,
Laser diodes,
a Laser Diode Driver,
a divergent Light beam output,
a biological reporter system, and
a Beam combiner.

According to a preferred embodiment of the present invention, the device uses impedance spectroscopy and analyzing it to produce the diagnostic profile of an individual's health and using initial treatments to see how the profile change and then adjusting the treatment parameters field for improved outcome.

According to a preferred embodiment of the present invention, the invention is a device used for initial treatment at 42.2 Gigahertz, which leads to Alu sequence decompaction and activation of the DNA improved biological outcomes, improved the coherence and orderliness of the DNA and of the DNA fractal patterns. Moreover, we have suggested that the main function of Alu is vibrational and proposed that Alu is responsible for the creation of the uniquely human (primate) morphogenic field and that is the key resonating component of our mind and consciousness. Also, we believe that Alus are creating the main part of the field in the nucleus and while interacting with the field they make the major contribution in the control of which genes are transcribed.

According to a preferred embodiment of the present invention, the DNA sequence-specific activation and/or repression of certain DNA sequences is achieved.

According to a preferred embodiment of the present invention, the device is used for application to therapy, communication to and from the body, communication to and from the brain, biotechnology and biological research. On communication to and from the body, referring to FIG. 1, it was proposed that the microtubules (101) and DNA communicate resonantly through the nuclear membrane (102) and that the microtubules of neighboring cells communicate (104) resonantly through the contact points of the cell membranes (103), thereby integrating all the body nuclei by the waveguides. From this, it follows that resonant vibrations of DNA propagate not by chance, but are guided by waveguides of microtubules. It was also proposed that the nervous system and connective tissue are primarily responsible for uniting the organism into one resonating system.

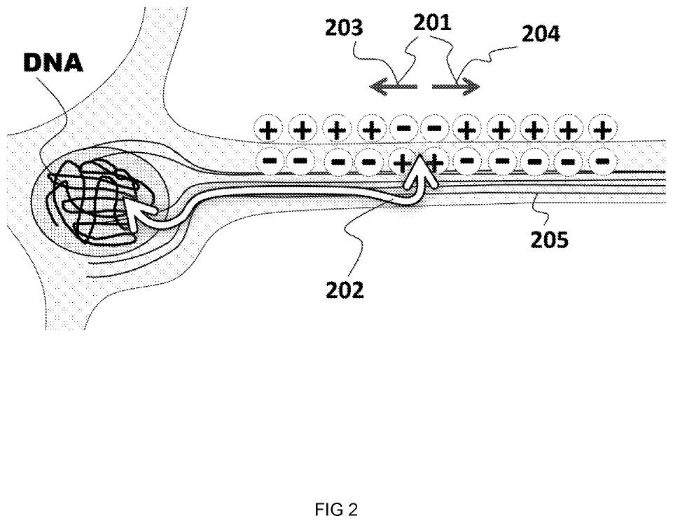

Referring to FIG. 2, it was also proposed that in neurons (203, 204), the propagation of action potential (201) across the axons is the process of reading and writing of information into and from microtubules (205) and that this propagation of action potential is electromagnetically connected (202) via the microtubules with the DNA in the nucleus thus allowing the participation of DNA in the work of mind and memory.

According to a preferred embodiment of the present invention, the method, and system where the sequential signals are patterned in time.

According to a preferred embodiment of the present invention the method and system where spatially patterned signals are produced. Spatially patterned signals, the shape of the wave, which is its circular polarization, chirality, and its 3-dimensional structure.

According to a preferred embodiment of the present invention the method and system of obtaining the patterns via bio-impedance spectroscopy, analyzing them, interpreting and returning to the body as DNA resonance treatment, in accordance with the obtained information.

According to a preferred embodiment of the present invention is a method and system of obtaining an electromagnetic signature from DNA samples, or living tissues or living bodies and recording it, amplifying and returning back to activate or repress specific DNA sequence groups for therapy, biotechnology, and other applications.

Moreover, another embodiment of this invention is the process of starting the treatment, then observing the changes via DNA resonance diagnostics and adjusting the treatment accordingly to improve the diagnostic results; which can be done in real-time or in alternating steps during treatment and diagnostic.

Finally, another unique embodiment of this invention is the method where biofeedback is based on electric measurements, electromagnetic measurements, sound measurements, temperature measurements, optical measurements, optoacoustic or electroacoustic measurements, measurements of temperature, detailed pulse characteristics, bio-impedance spectroscopy, a response of the above measurements to active perturbation, and/or measurements performed at specific points including acupuncture points and Zakharyin-Ged Zones.

In the device as shown in FIG. 3, the computer with a National Instruments Analog I/O PCR card was used to control the laser diode driver. The laser diode driver was used to power two laser diodes: a 660 nm red LED and an 880 nm NIR (near-infrared) LED. The light from the laser diodes was combined using a fiberoptic beam combiner. The divergent light beam coming out of the beam combiner was directed to a biological sample, which was a cell culture in a petri dish. A custom program in a LabView platform was created to run the irradiation in one of the two modes: ALT (alternated) and SYN (synchronized), Fig. [Modes]. In each of the two modes, the light of each of the two wavelengths was pulsed for 1 msec (millisecond) followed by a pause for 1 msec. In ALT mode, the pulses of the two wavelengths were alternated in time: red pulse, NIR pulse, red pulse, NIR pulse. So there was always a flux of light, only the wavelength was changed every 1 msec. In the SYN mode, the pulses and pauses of the two wavelengths were synchronized, so there was a simultaneous pulse of combined red and NIR wavelengths, followed by a pause, FIG. 5.

The biological experiment was done as follows: An adherent C3H mouse fibroblast culture (C3H/10T1/2, Clone 8 ATCC CCL-226) was grown in four Petri dishes to 70% confluency.

The treatments were as follows:
Dish1—ALT mode—sample ALT1
Dish2—ALT mode—sample ALT2
Dish3—SYN mode—sample SYN1
Dish4—no light mode—sample NLM1

All samples were done in quadruplicates. Each dish was treated at pulse fluence 9.7 J/cm2 for each wavelength for a total of 15 min. Dishes 1 and 2 were subjected to irradiation in ALT mode. The Dish3 was irradiated in the SYN mode. Therefore all irradiated dishes received the same dose of each wavelength and the pulses were at the same frequency, except in dishes 1 and 2, the pulses were in the alternating pulsing phases and in the Dish3 the pulses were in the same pulsing phase (synchronized).

According to our model, the ALT mode should have an activating effect on genes with certain sequence patterns in their promoters. The dishes were incubated for 3 hours to allow for gene activation and expression and the RNA was extracted. Gene expression was compared between pairs of samples using a 20,000 gene Illumina gene expression microarray as follows:

Comparison1: ALT2 over NLM1 (to determine which genes are activated relative to no light mode)

Comparison2: ALT1 over SYN1 (to determine the effect of the wavelength pulse alternation)

A subset of 289 genes was selected which were activated over 2 fold in ALT2 over NLM1 (Comparison1). The genes in this subset were ranked by the preference of expression in ALT1 over SYN1 (Comprison2). As a result, a gene Cdc25a was found to have the highest fold activation in both comparisons. We inspected the sequence of Cdc26a promoter for tandems and found in it two tandems: (GA)28 located 231-287 bp (base pairs) and (GGGA)8 located 411-443 bp upstream of the transcription start of the Cdc25a gene and separated by a gap of 124 bp. To verify the preferential activation of the Cdc25a promoter by ALT over SYN and NLM, we purchased a plasmid containing luciferase driven by a CDC25a promoter from SwitchGear Genomics, Menlo Park, Calif. The cells were transfected with the plasmid, subjected to the same treatments and the same comparisons were repeated using a Sigma-Aldrich Luciferase assay and quantified using a plate reader. The analyses were done in quadruplicates. The construct demonstrated up to 6.7-fold preferential expression in ALT over SYN mode (p<0.05 by t-test) while the expression in NLM mode was negligible. Two reference promoters used as negative controls didn't show preferential expression in ALT over SYN mode. Therefore, the preferential activation of the Cdc25a promoter by ALT over SYN mode was confirmed in an independent experiment.

This result proved the principle of SRAD. A specific gene was shown to be activated by a specific multiwavelength pattern and these parameters are mechanistically explained by the mechanistic SRAD model, FIG. 4 describes the sequence-specific activation of oscillations by the alternating pulses of unfocused 660 nm red and 850 nm NIR light in Cdc25a promoter containing (GGGA)8 and (GA)28 tandem repeats, FIG. 4 [Model]A. The oscillations are torsional, they occur by elastic twisting of parts of the double helix around its axis, FIG. 4 [Model] G and H. To illustrate these oscillations we flattened the surface of the cylinder, wrapped around the double helix (FIG. 4 [Model] G and H) and showed its normal undisturbed relaxed B-form state as a central horizontal line in FIG. 4 [Model] B. The oscillations are induced in the promoter by alternating pulses of 660 nm red and 850 nm infrared light as described above. During the first pulse of 850 nm light, (GGGA)8 tandem repeat sequence specifically absorbs the light as it is its own natural oscillation frequency, the energy causes its temporary warming up and unwinding (as DNA unwinds by warming up). When the pulse is gone, the DNA returns to its relaxed state. The unwinding of the (GGGA)8 results in a spike in FIG. 4 [Model] C: the horizontal line in (GGGA)8 region tilts counterclockwise. This mechanically turns a fragment of DNA immediately downstream of (GGGA)8 around its axis as shown by a peak downstream of (GGGA)8 FIG. 4 [Model] C. Similarly, this creates a trough upstream of (GGGA)8, FIG. 4 [Model] C. Next a pulse of 660 nm red light warms up and unwinds (GA)28 tandem repeat creating a similar unwinding pattern, FIG. 4 [Model] D. A combination of images (B) and (C) allows to see the rotational movement of the DNA fragment between (GGGA)8 and (GA)28, FIG. 4 [Model] E. Furthermore, repetitive alternation of pulses of 660 nm red and 850 nm NIR light every 1 millisecond creates propagation of twisting waves outside of the tandem repeats (GGGA)8 and (GA)28, FIG. 4 [Model] F. These waves reach Cdc25a transcription start, warm it up and this way facilitate the dissociation of the DNA strands in the transcription start and activation of transcription (not shown). A third-dimensional representation of such waves is shown a 3D projection of twisting oscillation. FIG. 4 [Model] H. While the DNA remains double-helical FIG. 4 [Model] (G), its twisting oscillates between the solid curve and the broken curve as shown in FIG. 4 [Model] H.

Unless otherwise defined, all terms (including technical terms) used herein have the same meaning as commonly understood by one having an ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The terminology used herein is for the purpose of describing particular embodiments only and is not intended to be limiting of the invention. As used herein, the term includes any and all combinations of one or more of the associated listed items. As used herein, the singular forms are intended to include the plural forms as well as the singular forms, unless the context clearly indicates otherwise.

While the preferred embodiments of the present invention has been shown and described in detail, it is to be understood that numerous modifications can be made to the preferred embodiment without departing from the spirit of the invention. Obviously, numerous variations and modifications can be made without departing from the spirit of the present invention. Therefore, it should be clearly understood that the forms of the present invention described above and shown in the figures of the accompanying drawings are illustrative only and are not intended to limit the scope of the present invention.

In describing the invention, it will be understood that a number of techniques are disclosed. Each of these has individual benefits and each can also be used in conjunction with one or more, or in some cases all, of the other disclosed techniques. Accordingly, for the sake of clarity, this description has refrained from repeating a possible combination in an unnecessary fashion. Nevertheless, the specifications and claim/s should be read with the understanding that such combinations are entirely within the scope of the invention and the claim/s.

What is claimed is:

1. Method and system of DNA resonance and its applications, the system comprising of
a computer,
a Signal generator,
a Laser Diode Driver,
Laser diodes,
a divergent Light beam output,
a Beam combiner,
and a biological system
characterized in that the system non-destructively affects DNA and/or receives information from DNA in live cells via non-chemical means.

2. Method and system as claimed in claim 1 wherein the method includes obtaining an electromagnetic signature from DNA samples, or living tissues or living bodies and recording it, amplifying and returning back to activate or repress specific DNA sequence groups for therapy, biotechnology, and other applications.

3. Method and system as claimed in claim 1 wherein the method includes obtaining the patterns via bio-impedance spectroscopy, analyzing them, interpreting and returning to the body as DNA resonance treatment, in accordance with the obtained information.

4. Method and system as claimed in claim 1 wherein the method includes a process of starting the treatment, then observing the changes via DNA resonance diagnostics and adjusting the treatment accordingly to improve the diagnostic results; which can be done in real-time or in alternating steps during treatment and diagnostic.

5. Method and system as claimed in claim 1 wherein the method includes application of certain specific frequencies and waveforms of electric current, electromagnetic, magnetic, optical, infrasound, audible sound, ultrasound or other forms of waves will induce a resonance condition in the DNA and related biological structures.

6. Method and system as claimed in claim 1 wherein characteristics of the waveform(s) (i.e., amplitude, frequency, phase, polarization, rotary polarization, waveform, etc) may be varied in order to affect the resonant condition so induced.

7. Method and system as claimed in claim 1 wherein the method includes the relative mix of two or more waveforms and phase-coordinated patterned combinations of thereof which may be varied so as to affect the resonant condition in order to achieve one or more desired outcomes.

8. Method and system as claimed in claim 1 wherein a biometric feedback signal is detected, where such signal is responsive to the applied Waveform(s), and where such creates a feedback loop such that Waveform(s) may be automatically (and/or manually) varied in response to such signal in order to achieve the desired outcome.

9. Method and system as claimed in claim 1 wherein biometric feedback (biofeedback) is based on electric, electromagnetic, acoustic, thermal, optical, optoacoustic or electroacoustic measurements, detailed pulse characterization, bioimpedance spectroscopy, and a response of the above measurements to artificial perturbation.

10. Method and system as claimed in claim 1 wherein biofeedback is based on electric measurements, electromagnetic measurements, sound measurements, temperature measurements, optical measurements, optoacoustic or electroacoustic measurements, measurements of temperature, detailed pulse characteristics, bio-impedance spectroscopy, a response of the above measurements to active perturbation, and/or measurements performed at specific points including acupuncture points, reflexology points, and Zakharyin-Ged Zones.

11. Method and system as claimed in claim 1 and working thereof wherein the DNA sequence-specific activation and/or repression of certain DNA sequences are achieved.

12. Method and system as claimed in claim 1 and working thereof wherein one or more of electric current, electromagnetic waves, electric voltage varied in time, magnetic pulses, infrasound, audible sound, ultrasound or other such means of stimulation applied individually and/or in combinations are used.

13. Method and system as claimed in claim 1 and working thereof wherein sequential signals are being patterned in time.

14. Method and system as claimed in claim 1 and working thereof wherein spatially patterned signals are produced. Spatially patterned signals, the shape of the wave, which is its circular polarization, chirality, and its 3-dimensional structure.

15. Method and system as claimed in claim 14 and working thereof wherein spatially patterned waves are combined and their phases are coordinated.
